(19) United States
(12) Patent Application Publication
(10) Pub. No.: US 2016/0361023 A1
(43) Pub. Date: Dec. 15, 2016

(54) TECHNIQUES FOR DETERMINING PHYSIOLOGICAL PROPERTIES OF A USER USING VASCULAR-RELATED SIGNALS QUALIFIED BY ACTIVITY STATE

(71) Applicant: QUALCOMM Incorporated, San Diego, CA (US)

(72) Inventors: Russel Allyn Martin, Menlo Park, CA (US); Ramin Samadani, Menlo Park, CA (US)

(21) Appl. No.: 14/736,115

(22) Filed: Jun. 10, 2015

(51) Int. Cl. A61B 5/00; A61B 5/11; A61B 5/021; A61B 5/024

(52) U.S. Cl. CPC A61B 5/7221; A61B 5/02416; A61B 5/1118; A61B 5/7278; A61B 5/021; A61B 2562/0219

(57) ABSTRACT

Techniques for determining one or more physiological properties of a user of a device is disclosed. The techniques include, in part, obtaining one or more vascular-related signals and a first set of data corresponding to one or more inertial sensors. The one or more vascular-related signals and the first set of data correspond to a common time interval. The techniques further include determining one or more motion state categories in accordance with the first set of data, selecting portions of the one or more vascular-related signals based on their corresponding motion state category, and processing the selected portions of the one or more vascular-related signals to determine the physiological properties of the user.

### TECHNICAL FIELD

Aspects of the disclosure relate to mobile devices, and more particularly, a system and method for determining one or more physiological properties of a user operating a mobile device.

### BACKGROUND

In photoplethysmography (PPG), signals corresponding to heart beats of a user are measured using one or more PPG sensors. In general, a wearable or portable device may be equipped with PPG sensors and/or processing units. This enables continuous monitoring of a user's heart rate or heart rate variability. However, motion of the user can affect quality and/or accuracy of the measured PPG signals.

### BRIEF SUMMARY

In one example, a method for determining one or more physiological properties of a user of a device is disclosed. The method includes, in part, obtaining one or more vascular-related signals and a first set of data corresponding to one or more inertial sensors. The one or more vascular-related signals and the first set of data correspond to a common time interval. The method further includes determining one or more motion state categories in accordance with the first set of data, selecting portions of the one or more vascular-related signals based on their corresponding motion state category, and processing the selected portions of the one or more vascular-related signals to determine the physiological properties of the user.

In one example, the selected portions of the one or more vascular-related signals correspond to a plurality of discontinuous portions of the common time interval. In one example, the selected portions of the one or more vascular-related signals correspond to a plurality of continuous portions of the common time interval. The method further includes, in part, determining one or more weights corresponding to the one or more motion state categories, and processing the selected portions of the one or more vascular-related signals in accordance with the one or more weights.

In one example, the method further includes determining a total duration of the selected portions of the one or more vascular-related signals corresponding to a first motion state category is less than a threshold, and causing a power level associated with the one or more vascular-related signals to be increased upon determination that the total duration of the selected portions corresponding to the first motion state category is less than the threshold. In one example, the one or more inertial sensors comprise an accelerometer.

In one example, the one or more vascular-related signals comprise a photoplethysmography (PPG) signal. In one example, the selected portions of the one or more vascular-related signals correspond to a common motion state category. In one example, the one or more physiological properties of the user comprise a heart rate, blood pressure or any other physiological property.

In one example, an apparatus for determining one or more physiological properties of a user is disclosed. The apparatus includes, in part, at least one processor and a memory coupled to the at least one processor. The at least one processor is configured to obtain one or more vascular-related signals and a first set of data corresponding to one or more inertial sensors. The one or more vascular-related signals and the first set of data correspond to a common time interval. The one or more processor is further configured to determine one or more motion state categories in accordance with the first set of data, select portions of the one or more vascular-related signals based on their corresponding motion state category, and process the selected portions of the one or more vascular-related signals to determine the physiological properties of the user.

In one example, an apparatus for determining one or more physiological properties of a user is disclosed. The apparatus includes, in part, means for obtaining one or more vascular-related signals and a first set of data corresponding to one or more inertial sensors, wherein the one or more vascular-related signals and the first set of data correspond to a common time interval, means for determining one or more motion state categories in accordance with the first set of data, means for selecting portions of the one or more vascular-related signals based on their corresponding motion state category, and means for processing the selected portions of the one or more vascular-related signals to determine the physiological properties of the user.

In one example, a non-transitory processor-readable medium for determining one or more physiological properties of a user is disclosed. The non-transitory processor-readable medium includes, in part, processor-readable instructions configured to cause one or more processors to obtain one or more vascular-related signals and a first set of data corresponding to one or more inertial sensors, wherein the one or more vascular-related signals and the first set of data correspond to a common time interval, determine one or more motion state categories in accordance with the first set of data, select portions of the one or more vascular-related signals based on their corresponding motion state category, and process the selected portions of the one or more vascular-related signals to determine the physiological properties of the user.

### BRIEF DESCRIPTION OF THE DRAWINGS

Aspects of the disclosure are illustrated by way of example. In the accompanying figures, like reference numbers indicate similar elements.

FIG. 1 illustrates a smartphone device configured to obtain vascular-related signals from a user, according to one embodiment of the present disclosure.

FIG. 2 illustrates a cross sectional view of the wrist worn device configured to obtain vascular-related signals from a user and graphs showing measurements obtained by the wristwatch device, according to one embodiment of the present disclosure.

FIG. 3 illustrates example operations which may be performed by a device for determining one or more physiological properties of a user of the device, according to one embodiment of the present disclosure.

FIGS. 4A and 4B illustrate example heart rate measurements and signal quality metrics, according to one embodiment of the present disclosure.

FIG. 5 illustrates example operations which may further be performed by the device to determine the physiological properties of the user, according to one embodiment of the present disclosure.

FIG. 6 illustrates example operations which may further be performed by the device to determine the physiological properties of the user, according to one embodiment of the present disclosure.

FIG. 7 is a flow diagram illustrating a plurality of derived physiological properties from a plurality of sensor measurements, according to one embodiment of the present disclosure.

FIG. 8 illustrates an example of a computing system in which one or more embodiments may be implemented.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure or the spirit of the appended claims.

Certain embodiments determine one or more physiological properties of a user based on vascular-related signals that are measured by one or more sensors. The term “vascular-related signals” is used herein to refer to any signal that is associated with beating of a user's heart, their respiration and the state of their vascular system. The vascular-related signals may be measured using different methods, such as photoplethysmography (PPG), Impedance plethysmography (IPG), ultrasound, radiofrequency reflection measurement, and the like. For example, in PPG technique, optical reflections off a user's blood vessels are measured. In IPG, a current is run through the tissue and changes in impedance are measured. In ultrasound, acoustical reflection is measured to determine vascular-related signals. In radiofrequency reflection measurement, reflections of a radio frequency signal, such as Radar off of a user's tissues are measured. In general, vascular-related signals may be measured using any known techniques without departing from the teachings of the present disclosure.

Motion of the user can affect quality of the vascular-related signals in several ways. For example, in PPG technique, motion of the measuring device relative to the user's skin can change the amount of light reflected from the user's tissues. Motion of user's body parts, such as arms can cause relative motion of muscles, tendons and blood vessels and thus change the PPG signal. In addition, movement of the measuring device can let in different amounts of ambient light and affect the PPG signal.

Several techniques exist in the art for improving mechanical and optical design of the measuring device to reduce the noise and/or imperfections in the PPG signals. Another technique is using an accelerometer in the measuring device. The accelerometer provides a measure of the motion, which is correlated with the amount of noise in the PPG signal. Measures of motion may be used to reduce the amount of noise in the PPG signal. However, these methods are not able to remove all the noise from the measured signals. The noise is particularly problematic in some applications, such as determining heart rate variability. In heart rate variability analysis, every beat of the heart must be accurately characterized. Therefore, averaging in time for the purpose of noise reduction is not possible. Although most of the examples described in this document are directed to measurement of PPG signals, it should be noted that the methods described herein may be used to qualify any type of signal, without departing from the teachings of the present disclosure.

Most of the work in the area of noise reduction in measured signals has been directed toward continuous filtering of the signals. However, removal of noise from a PPG signal by filtering and/or utilizing the measurements from the accelerometer has limits.

According to one embodiment, physiological state of a user may be determined even if a valid signal is not present all the time. In one embodiment, portions of the signal that has minimal or no noise is identified and used to determine the physiological state of the user. It should be noted that many physiological signals include valuable information, even if they are only measured occasionally. Certain embodiments qualify one or more portions of a measured signal that correspond to limited amount of noise. For example, in one embodiment, a first portion of the vascular related signal and a second portion of the vascular related signal are used in determining the physiological state of a user. In this example, data corresponding to a third portion of the vascular-related signal is not used. The third portion of the vascular-related signal may correspond to an amount of noise higher than a threshold. Certain embodiments use an activity state of the user to determine if the measured signal is acceptable or not. For example, if the user is resting, the measured signal may include smaller amount of noise that can be used to determine physiological properties of the user. On the other hand, if the user is running, the measurements from the PPG sensors may not be very accurate, and therefore will not be used to determine the physiological properties of the user.

The activity state may be determined using well-known motion classification methods in the art. For example, measurements from a body-worn accelerometer may be used to classify motion of the user. For example, activity trackers (e.g., the Fitbit) determine different categories of motion, such as running, walking, cycling, inactive, sleeping based on the readings from the accelerometers. In some software packages motion categories are displayed on a timeline with physiological measurements. These activity trackers use activity state as an input into measures of calories expended and may include other sensors beyond accelerometer. For example, they may use barometric pressure to measure if the subject is going up stairs and modify the calories expended accordingly.

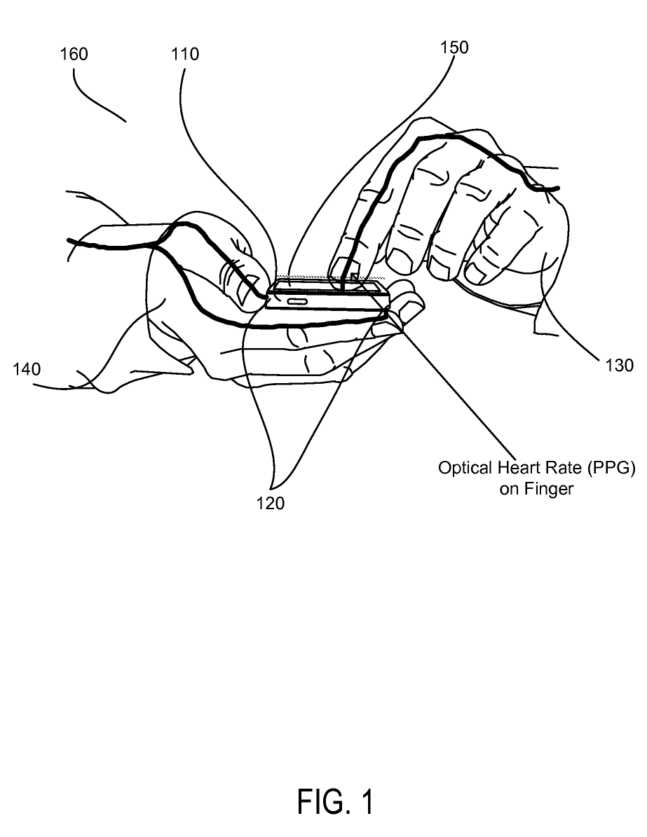

FIG. 1 illustrates a smartphone device 110 configured to obtain PPG measurements of a user, according to some embodiments. It can be appreciated that the smartphone device 110 is only one example of device capable of obtaining physiological measurements of the user. The smartphone device 110 may include a plurality of contacts 120. In some embodiments, a single contact 120 may be positioned at each end of the smartphone device 110. In other embodiments, a device front surface 150 of the smartphone device 110 may include a contact layer including, e.g., silver metal or Indium Tin Oxide (ITO). The smartphone device 110 obtains physiological signals (e.g., vascular-related signals) corresponding to the user 160 through one or more sensors. In some embodiments, the device front surface 150 may be a touchscreen.

For example, the user 160 may hold the smartphone device 110 with his/her first hand 140 touching one or more of the contacts 120 and with his/her second hand 130 touching the device front surface 150. The device front surface 150 of the smartphone device 110 may obtain a PPG measurement of the user 160 by using an optical-based technology. For example, when the user 160 touches the device front surface 150, the touchscreen may shine a light into the user's 160 skin through a light source, measure the blood flow through the capillaries using one or more sensors, and thus determine a heart rate of the user. This process is described in further detail below.

It can be appreciated that front surface 150 of the device may serve multiple functions. That is, front surface 150 of the device may be used to obtain PPG and/or other physiological measurements, and may also be used as a user input device. The user 160 may use the device front surface 150 to provide input to applications being executed on the smartphone device 110. When the user 160 wishes to obtain a bodily function measurement using the device front surface 150, the user 160 may place the smartphone device 110 into a measurement mode. Alternatively, the smartphone device 110 may automatically detect the user's intention to obtain a bodily function measurement, e.g., from the user 160 placing his/her finger in a particular location on the device front surface 150 or touching the device front surface 150 for a predetermined period of time. Alternatively, the smartphone device 110 may regularly scan and store vital signs of the user 160 in the user's normal course of operating the device 110, without the user wanting or requesting a particular vital sign report at that time.

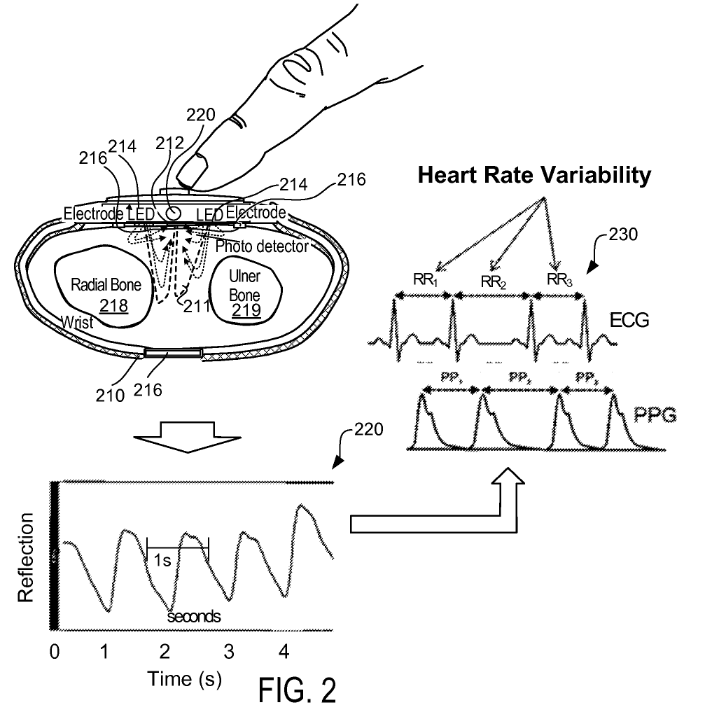

FIG. 2 illustrates a cross sectional view of the wristwatch 210 configured to obtain PPG and/or other vascular-related signal measurements corresponding to a user. In addition, graphs 220, and 230 show measurements obtained by the wristwatch device, according to some embodiments. The wrist worn device 210 operates similarly to the smartphone device 110 in FIG. 1. That is, the wrist worn device 210 may obtain PPG, and other signal measurements of the user 160 via a plurality of contacts. In some embodiments, one or more contacts may be placed at the bottom of the wrist worn device 210, where the contact makes a continuous contact with the user's wrist while the user 160 wears the wrist worn device 210.

The cross sectional view of the wrist worn device 210 shows a photodetector 212, a plurality of light emitting diodes (LED) 214, and a plurality of electrodes contacts 216. Additionally, the cross sectional view 210 also illustrates parts of a user's wrist, e.g., radial bone 218 and ulnar bone 219. The wrist worn device 210 may also include a multifunction button 220 which may be used to obtain a signal measurement and also as a user input device. For example, the multifunction button 220 may be used by the user 160 to set a date and/or time for the wrist worn device 210. The PPG measurements may be obtained in a similar fashion as described with respect to the smartphone device of FIG. 1, e.g., via the contacts and/or multi-function button 220 on the wrist worn device 210.

The photodetector 212 may be physically coupled to the outer body of the wrist worn device 210 and be configured to obtain data. Light emitting diodes (LED) 214 are configured to emit light through a user's body. The LEDs 214 are typically positioned at the bottom of the wrist worn device 210 and on top of the user's wrist. The emitted light may be of a wavelength that can pass through parts of a user's body. For example, the LEDs may emit light through a user's wrist. The light emitted from light source may reflect off of or pass through blood vessels within the user's body and the reflected or transmitted light may be measured by one or more photodetectors 212 to obtain a PPG measurement. The user's blood volume may be determined based off of the reflected or transmitted light as compared against time. From these data, the user's PPG measurement may be determined. In some embodiments, the determination of the user's local blood volume may be determined from a change in the user's blood vessels. More specifically, a change in the diameter of the blood vessels that are being probed by the LEDs 214.

It can be appreciated that emitted light may be of different wavelengths. For example, different wavelengths of light may be appropriate to improve the signal, reduce noise, deal with dark skin colors, measure the blood's oxygen content, or penetrate to different depths of the user's body.

It can be appreciated that the outer body of the wristwatch 210 may be sized to be portable for a user. It can be appreciated that the term “portable” may refer to something that is able to be easily carried or moved, and may be a light and/or small. In the context of embodiments of the present invention, the term portable may refer to something easily transportable by the user or wearable by the user. For example, the smartphone device 110 or the wristwatch 210 may be examples of portable devices. Other examples of portable devices include a head-mounted display, calculator, portable media player, digital camera, pager, earpiece, personal navigation device, etc. Examples of devices that may not be considered portable include a desktop computer, traditional telephone, television, appliances, etc.

In some embodiments, the wrist worn device 210 may perform everyday functions other than obtaining physiological measurements of the user. For example, the wrist worn device 210 may provide the current time, a stopwatch function, a calendar function, communication functions, etc. The PPG, heart rate, blood pressure, respiration rate and other measurements may be available in addition to the other described functions on the wrist worn device 210.

Graph 220 illustrates the intensity of the obtained light reflections at the photodetector 212 against time. In this example, the duration between each pulse is approximately one second. From this graph, the user's PPG can be determined Graph 230 shows a user's heart rate variability by comparing different vascular-related signals (e.g., ECG and PPG) that are measured by the device.

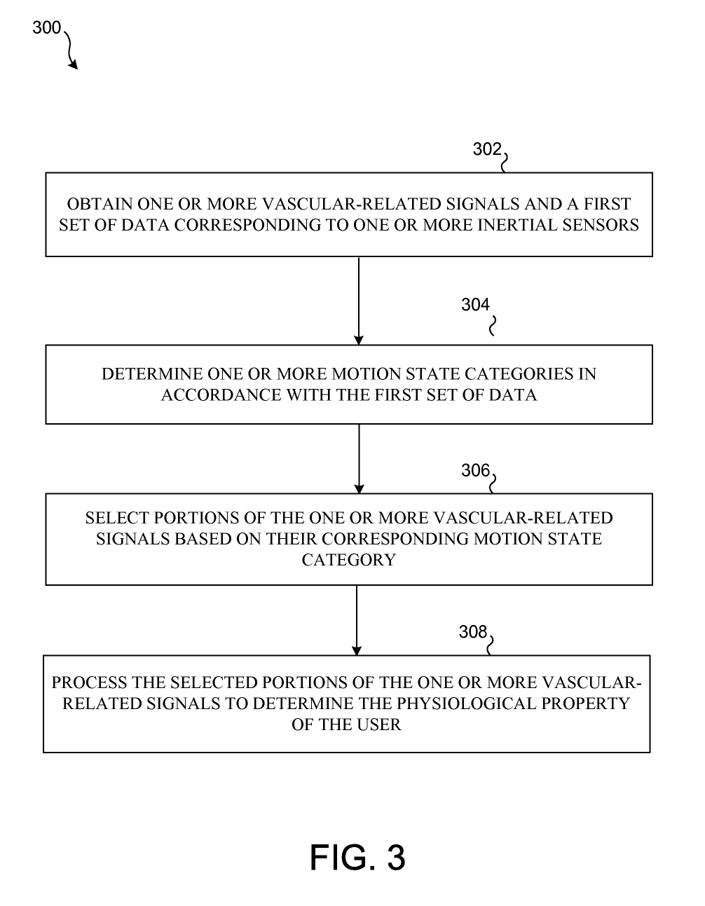

FIG. 3 illustrates example operations which may be performed by a device to determine one or more physiological properties of a user of the device. At 302, the device obtains one or more vascular-related signals (e.g., PPG signal) and a first set of data corresponding to one or more inertial sensors. The one or more vascular-related signals and the first set of data correspond to a common time interval. The inertial sensors may include one or more accelerometers, barometers, gyroscope, or any other type of environmental and/or inertial sensors.

At 304, the device determines one or more motion state categories in accordance with the first set of data. For example, the device determines a resting state, a walking state and a running state for the user based on the readings from the inertial sensors.

At 306, the device selects a portions of the one or more vascular-related signals based on their corresponding motion state category. In one embodiment, the selected portions of the one or more vascular-related signals correspond to a common motion state category. For example, the device may select portions of the vascular-related signals that correspond to the resting state of the user.

At 308, the device processes the selected portions of the one or more vascular-related signals to determine the one or more physiological properties of the user. In one embodiment, the physiological properties of the user may include a heart rate, hear rate variability, blood pressure, or any other physiological properties.

In one embodiment, a motion classifier is used to determine if the measured physiological signals are valid or not. For example, when PPG signals are measured at the wrist to determine heart rate variability, measurements from an accelerometer in the same package as the PPG sensor can be used to determine if the arm was relatively still during the PPG signal measurements. If the arm was still, the PPG signal is much cleaner and more reliable. Additionally, the DC level of the PPG signal can be used to detect changes, for example from movement of tendons within an arm.

In one embodiment, the data may continuously be collected from the PPG sensor. However, result of heart rate variability determination may only be shown to the user when there has been at least a predefined time interval in which the user's arm is still enough that the signal is clean. In general, in order to correctly determine heart rate variability, at least the predefined time interval (e.g., two minutes) of continuous data may need to be collected to get some of the more valuable metrics, such as low frequency (LF), high frequency (HF), and/or their ratio. Longer periods of valid signals may result in more reliable metrics corresponding to physiological properties of the user.

In one embodiment, the selected portions of the one or more vascular-related signals correspond to a plurality of discontinuous portions of the common time interval. For example, it is possible to assemble pulse to pulse times from a number of shorter periods (e.g., discontinuous portions) where the arm is still and get reasonable values.

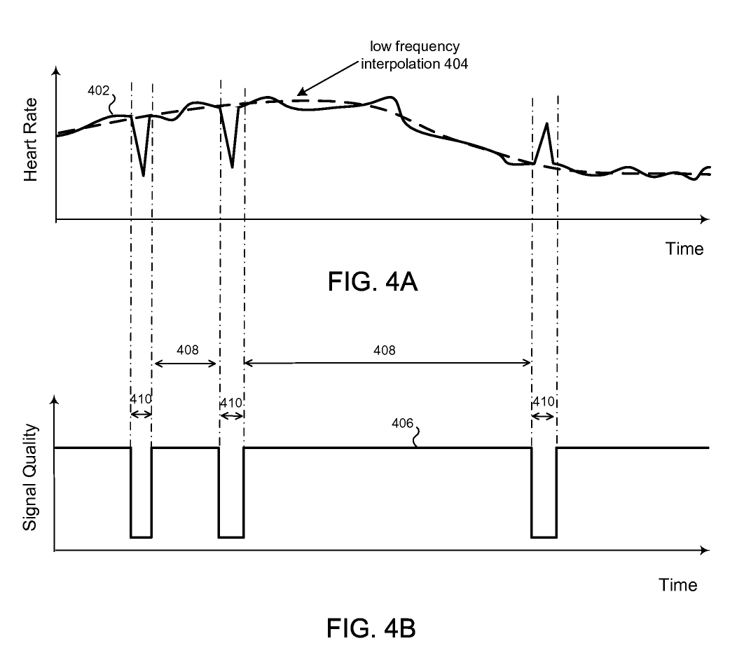

FIGS. 4A and 4B illustrate example heart rate measurements and signal quality metrics, according to one embodiment of the present disclosure. Curve 402 in FIG. 4A, illustrates heart rate measurements over time. Curve 404 shows a low-frequency interpolation 404 of the heart rate measurements. As illustrated in FIG. 4B, signal quality metric 406 indicates that in some periods of time (e.g., period 408) quality of signal is high, and in some other periods (e.g., periods 410), quality of signal is low. According to one embodiment, the heart rate signal that is measured during low quality periods (e.g., periods 410) may be discarded, and the discontinuous portions corresponding to high quality periods may be selected for further analysis. However, it should be noted that these shorter periods (e.g., periods 410) may not be too far separated in time. Otherwise, the value of correlating physiological events to actual events would be lost. For example, if data corresponding to stress is collected over a two-hour time frame, it might not be possible to associate it with one event, such as a stressful conversation.

In one embodiment, the vascular-related signals may be analyzed in frequency domain. In one example, if the selected portions correspond to discontinuous portions of the vascular-related signals, the processing may include, low-pass filtering, resampling, anti-aliasing and/or any other processing techniques known in the art for determining the temporal properties of the signal. Interpolation can also be accomplished by fitting the signal to a functional form, such as a polynomial or a spline curve.

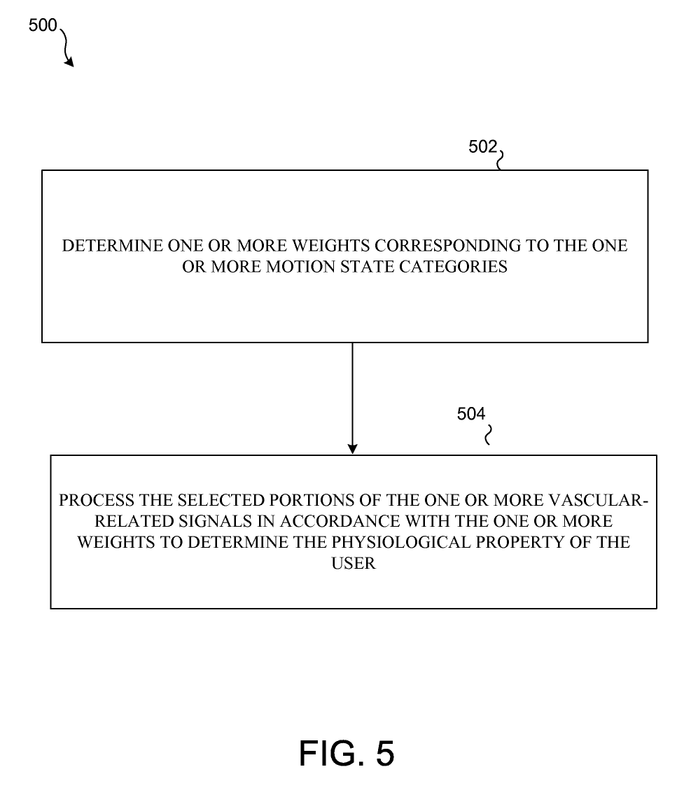

FIG. 5 illustrates example operations which may further be performed by the device to determine one or more physiological properties of the user. At 502, the device determines one or more weights corresponding to the one or more motion state categories. At 504, the device processes the selected portions of the one or more vascular-related signals in accordance with the one or more weights to determine one or more physiological properties of the user. For example, the device may assign a higher weight to the measurements that correspond to the resting state of the user, which may correspond to lower noise. In addition, the device may assign a lower weight to the measurements that correspond to the walking state of the user, since there may be extra noise caused by the movement of the hand/body, and the optical measurements of the PPG signals may not be accurate. In one embodiment, the weights may be assigned to continuous portions of the vascular-related signal.

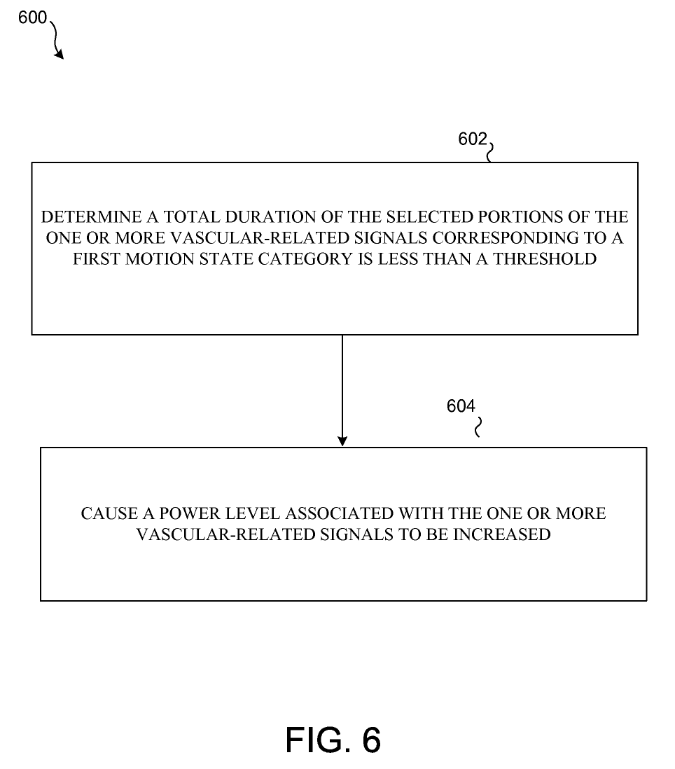

FIG. 6 illustrates example operations that may further be performed by the device to determine physiological properties of the user. At 602, the device may determine that a total duration of the selected portions of the one or more vascular-related signals corresponding to a first motion state category is less than a threshold. For example, the device may be interested in determining the physical property using the vascular-related measurements that correspond to the resting state of the user. If the device determines that the user is active and does not have enough resting time, at 604, the device may cause a power level associated with the one or more vascular-related signals to be increased. For example, in the case of measuring a PPG signal, the device may increase the power of the light source emitted into the skin, if the device determines that a total duration of the selected portions corresponding to the resting category is smaller than a threshold. In another example, the device may cause the power level associated with the one or more vascular-related signals to be increased if duration of one or more of the selected portions is less than a second threshold. In another example, the device may change the frequency of operation of the light source or make any other adjustments to increase signal to noise ratio of the measured vascular-related signal.

In one example, blood pressure is measured using a pulse transit time method. When the user stands up, a drop in blood pressure can be measured. In this case, the accelerometer may measure a signal of brief vertical acceleration followed by a stationary period. If the drop in blood pressure exceeds a threshold, the user can be alerted. The sitting to standing event could be distinguished from movement in an elevator by the length of the acceleration and by the combination of directions of the acceleration.

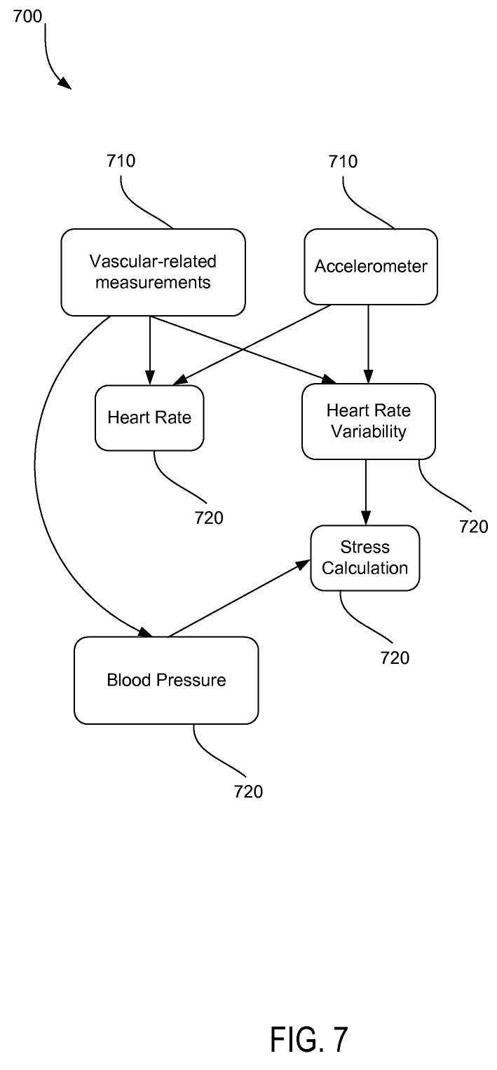

FIG. 7 is a flow diagram 700 illustrating a plurality of derived physiological properties 720 from a plurality of sensor measurements 710, according to some embodiments. The plurality of sensor measurements 710 may include, but is not limited to, vascular-related measurements, such as PPG pulse measurement, and motion measurements (e.g., accelerometer, gyroscope, and the like). These sensor measurements 710 may be obtained by taking measurements via the mobile device. Based on data from the sensor measurements 710, a plurality of physiological properties 720 may be derived. These physiological properties may include, but are not limited to, heart rate, heart rate variability, stress calculation, blood pressure, and the like.

For example, when a PPG pulse measurement is obtained, using the techniques described herein, the user's heart rate and/or heart rate variability may be determined. In one example, the PPG pulse measurements may be combined with other sensor measurements to determine the user's blood pressure. Based on the determined blood pressure, a user's stress level may be determined. If it is determined that the user is at a high stress level, the mobile device may notify the user to take a deep breath, go for a walk, drink a glass of water, etc.

In some embodiments, accelerometer measurements may also be used to determine when the vascular-related measurements have a high quality, which can then be used to determine the user's heart rate and/or heart rate variability. The same calculations described above may be determined/calculated using these measurements.

The previous description is provided to enable any person skilled in the art to practice the various aspects described herein. Various modifications to these aspects will be readily apparent to those skilled in the art, and the generic principles defined herein may be applied to other aspects. Moreover, nothing disclosed herein is intended to be dedicated to the public.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

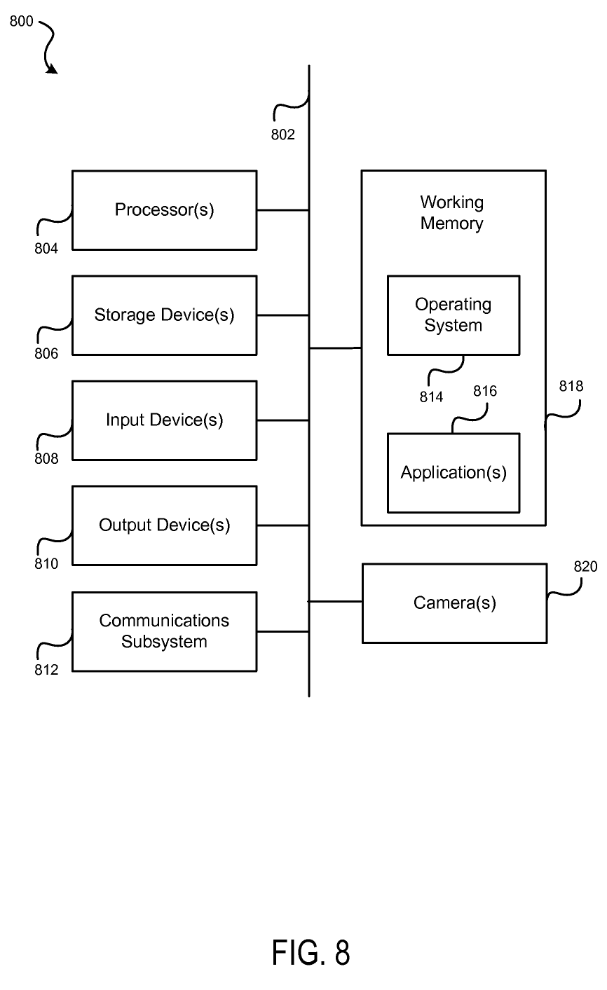

FIG. 8 illustrates an example of a computing system in which one or more embodiments may be implemented. A computer system as illustrated in FIG. 8 may be incorporated as part of the above described measurement device. For example, computer system 800 can represent some of the components of a watch, head-mount display, a laptop, desktop, tablet or any other suitable computing system. FIG. 8 is meant only to provide a generalized illustration of various components, any or all of which may be utilized as appropriate. FIG. 8, therefore, broadly illustrates how individual system elements may be implemented in a relatively separated or relatively more integrated manner. In some embodiments, elements of computer system 800 may be used to implement functionality of the mobile device 110 in FIG. 1 or wrist watch 210 in FIG. 2.

The computer system 800 is shown comprising hardware elements that can be electrically coupled via a bus 802 (or may otherwise be in communication, as appropriate). The hardware elements may include one or more processors 804, including without limitation one or more general-purpose processors and/or one or more special-purpose processors (such as digital signal processing chips, graphics acceleration processors, and/or the like); one or more input devices 808, which can include without limitation one or more sensors (e.g., sensors for measurement of vascular-related signals, inertial sensors, environmental sensors, etc.), a mouse, a keyboard, a microphone configured to detect ultrasound or other sounds, and/or the like; and one or more output devices 810, which can include without limitation a display unit such as the device used in embodiments of the invention, a printer and/or the like. The output devices may also include a light source that may be used to emit light into a user's skin to measure PPG signals.

The computer system 800 may further include (and/or be in communication with) one or more non-transitory storage devices 806, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data storage, including without limitation, various file systems, database structures, and/or the like.

The computer system 800 might also include a communications subsystem 812, which can include without limitation a modem, a network card (wireless or wired), an infrared communication device, a wireless communication device and/or chipset (such as a Bluetooth™ device, an 802.11 device, a Wi-Fi device, a WiMax device, cellular communication facilities, etc.), and/or the like. The communications subsystem 812 may permit data to be exchanged with a network, other computer systems, and/or any other devices described herein. In many embodiments, the computer system 800 will further comprise a non-transitory working memory 818, which can include a RAM or ROM device, as described above.

The computer system 800 also can comprise software elements, shown as being currently located within the working memory 818, including an operating system 814, device drivers, executable libraries, and/or other code, such as one or more application programs 816, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a computer-readable storage medium, such as the storage device(s) 806 described above. In some cases, the storage medium might be incorporated within a computer system, such as computer system 800. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computer system 800 and/or might take the form of source and/or installable code, which, upon compilation and/or installation on the computer system 800 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed. In some embodiments, one or more elements of the computer system 800 may be omitted or may be implemented separate from the illustrated system. For example, the processor 804 and/or other elements may be implemented separate from the input device 808. In some embodiments, elements in addition to those illustrated in FIG. 8 may be included in the computer system 800.

Some embodiments may employ a computer system (such as the computer system 800) to perform methods in accordance with the disclosure. For example, some or all of the procedures of the described methods in FIGS. 3 through 6 may be performed by the computer system 800 in response to processor 804 executing one or more sequences of one or more instructions (which might be incorporated into the operating system 814 and/or other code, such as an application program 816) contained in the working memory 818. Such instructions may be read into the working memory 818 from another computer-readable medium, such as one or more of the storage device(s) 806. Merely by way of example, execution of the sequences of instructions contained in the working memory 818 might cause the processor(s) 804 to perform one or more procedures of the methods described herein.

The terms “machine-readable medium” and “computer-readable medium,” as used herein, refer to any medium that participates in providing data that causes a machine to operate in a specific fashion. In some embodiments implemented using the computer system 800, various computer-readable media might be involved in providing instructions/code to processor(s) 804 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 806. Volatile media include, without limitation, dynamic memory, such as the working memory 818. Transmission media include, without limitation, coaxial cables, copper wire and fiber optics, including the wires that comprise the bus 802, as well as the various components of the communications subsystem 812 (and/or the media by which the communications subsystem 812 provides communication with other devices). Hence, transmission media can also take the form of waves (including without limitation radio, acoustic and/or light waves, such as those generated during radio-wave and infrared data communications).

In one embodiment, means for obtaining signals may include input devices 808 (e.g., sensors), or any other means that can be used to obtain, measure or receive these signals. Moreover, means for determining, means for selecting, means for processing, and means for causing may correspond to processor(s) 804 or any other means capable of performing these functions.

Common forms of physical and/or tangible computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to the processor(s) 804 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 800. These signals, which might be in the form of electromagnetic signals, acoustic signals, optical signals and/or the like, are all examples of carrier waves on which instructions can be encoded, in accordance with various embodiments of the invention.

The communications subsystem 812 (and/or components thereof) generally will receive the signals, and the bus 802 then might carry the signals (and/or the data, instructions, etc. carried by the signals) to the working memory 818, from which the processor(s) 804 retrieves and executes the instructions. The instructions received by the working memory 818 may optionally be stored on a non-transitory storage device 806 either before or after execution by the processor(s) 804.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A method for determining one or more physiological properties of a user of a device, comprising:
obtaining one or more vascular-related signals and a first set of data corresponding to one or more inertial sensors, wherein the one or more vascular-related signals and the first set of data correspond to a common time interval;
determining one or more motion state categories in accordance with the first set of data;
selecting portions of the one or more vascular-related signals based on their corresponding motion state category; and
processing the selected portions of the one or more vascular-related signals to determine the physiological properties of the user.

2. The method of claim 1, wherein the selected portions of the one or more vascular-related signals correspond to a plurality of discontinuous portions of the common time interval.

3. The method of claim 1, wherein the selected portions of the one or more vascular-related signals correspond to a plurality of continuous portions of the common time interval, and the method further comprises:
determining one or more weights corresponding to the one or more motion state categories; and
processing the selected portions of the one or more vascular-related signals in accordance with the one or more weights.

4. The method of claim 1, further comprising:
determining a total duration of the selected portions of the one or more vascular-related signals corresponding to a first motion state category is less than a threshold; and
causing a power level associated with the one or more vascular-related signals to be increased upon determination that the total duration of the selected portions corresponding to the first motion state category is less than the threshold.

5. The method of claim 1, wherein the one or more inertial sensors comprise an accelerometer.

6. The method of claim 1, wherein the one or more vascular-related signals comprise a photoplethysmography (PPG) signal.

7. The method of claim 1, wherein the selected portions of the one or more vascular-related signals correspond to a common motion state category.

8. The method of claim 1, wherein the one or more physiological properties of the user comprise a heart rate.

9. The method of claim 1, wherein the one or more physiological properties of the user comprise a blood pressure.

10. An apparatus for determining one or more physiological properties of a user of the apparatus, comprising:
at least one processor configured to:
obtain one or more vascular-related signals and a first set of data corresponding to one or more inertial sensors, wherein the one or more vascular-related signals and the first set of data correspond to a common time interval;
determine one or more motion state categories in accordance with the first set of data;
select portions of the one or more vascular-related signals based on their corresponding motion state category; and
process the selected portions of the one or more vascular-related signals to determine the physiological properties of the user; and
a memory coupled to the at least one processor.

11. The apparatus of claim 10, wherein the selected portions of the one or more vascular-related signals correspond to a plurality of discontinuous portions of the common time interval.

12. The apparatus of claim 10, wherein the selected portions of the one or more vascular-related signals correspond to a plurality of continuous portions of the common time interval, and the at least one processor is further configured to:
determine one or more weights corresponding to the one or more motion state categories; and
process the selected portions of the one or more vascular-related signals in accordance with the one or more weights.

13. The apparatus of claim 10, further comprising:
determine a total duration of the selected portions of the one or more vascular-related signals corresponding to a first motion state category is less than a threshold; and
cause a power level associated with the one or more vascular-related signals to be increased upon determination that the total duration of the selected portions corresponding to the first motion state category is less than the threshold.

14. The apparatus of claim 10, wherein the one or more inertial sensors comprise an accelerometer.

15. The apparatus of claim 10, wherein the one or more vascular-related signals comprise a photoplethysmography (PPG) signal.

16. The apparatus of claim 10, wherein the selected portions of the one or more vascular-related signals correspond to a common motion state category.

17. The apparatus of claim 10, wherein the one or more physiological properties of the user comprise a heart rate.

18. The apparatus of claim 10, wherein the one or more physiological properties of the user comprise a blood pressure.

19. An apparatus for determining one or more physiological properties of a user, comprising:
means for obtaining one or more vascular-related signals and a first set of data corresponding to one or more inertial sensors, wherein the one or more vascular-related signals and the first set of data correspond to a common time interval;
means for determining one or more motion state categories in accordance with the first set of data;
means for selecting portions of the one or more vascular-related signals based on their corresponding motion state category; and
means for processing the selected portions of the one or more vascular-related signals to determine the physiological properties of the user.

20. The apparatus of claim 19, wherein the selected portions of the one or more vascular-related signals correspond to a plurality of discontinuous portions of the common time interval.

21. The apparatus of claim 19, wherein the selected portions of the one or more vascular-related signals correspond to a plurality of continuous portions of the common time interval, the apparatus further comprising:
means for determining one or more weights corresponding to the one or more motion state categories; and
means for processing the selected portions of the one or more vascular-related signals in accordance with the one or more weights.

22. The apparatus of claim 19, further comprising:
means for determining a total duration of the selected portions of the one or more vascular-related signals corresponding to a first motion state category is less than a threshold; and
means for causing a power level associated with the one or more vascular-related signals to be increased upon determination that the total duration of the selected portions corresponding to the first motion state category is less than the threshold.

23. The apparatus of claim 19, wherein the one or more vascular-related signals comprise a photoplethysmography (PPG) signal.

24. The apparatus of claim 19, wherein the selected portions of the one or more vascular-related signals correspond to a common motion state category.

25. A non-transitory processor-readable medium for determining one or more physiological properties of a user, comprising processor-readable instructions configured to cause one or more processors to:
obtain one or more vascular-related signals and a first set of data corresponding to one or more inertial sensors, wherein the one or more vascular-related signals and the first set of data correspond to a common time interval;
determine one or more motion state categories in accordance with the first set of data;
select portions of the one or more vascular-related signals based on their corresponding motion state category; and
process the selected portions of the one or more vascular-related signals to determine the physiological properties of the user.

26. The non-transitory processor-readable medium of claim 25, wherein the selected portions of the one or more vascular-related signals correspond to a plurality of discontinuous portions of the common time interval.

27. The non-transitory processor-readable medium of claim 25, wherein the selected portions of the one or more vascular-related signals correspond to a plurality of continuous portions of the common time interval, and the processor-readable instructions are further configured to cause the one or more processors to:
determine one or more weights corresponding to the one or more motion state categories; and
process the selected portions of the one or more vascular-related signals in accordance with the one or more weights.

28. The non-transitory processor-readable medium of claim 25, wherein the processor-readable instructions are further configured to cause the one or more processors to:
determine a total duration of the selected portions of the one or more vascular-related signals corresponding to a first motion state category is less than a threshold; and
cause a power level associated with the one or more vascular-related signals to be increased upon determination that the total duration of the selected portions corresponding to the first motion state category is less than the threshold.

29. The non-transitory processor-readable medium of claim 25, wherein the one or more vascular-related signals comprise a photoplethysmography (PPG) signal.

30. The non-transitory processor-readable medium of claim 25, wherein the selected portions of the one or more vascular-related signals correspond to a common motion state category.
